(19) United States
(12) Patent Application Publication
(10) Pub. No.: US 2008/0243068 A1
(43) Pub. Date: Oct. 2, 2008

(54) METHODS AND APPARATUS FOR TREATMENT OF VENOUS INSUFFICIENCY

(76) Inventors: Kamal RAMZIPOOR, Fremont, CA (US); Ary CHERNOMORSKY, Walnut Creek, CA (US)

Correspondence Address: LEVINE BAGADE HAN LLP, 2483 EAST BAYSHORE ROAD, SUITE 100, PALO ALTO, CA, 94303, US

(21) Appl. No.: 11/461,764

(22) Filed: Aug. 1, 2006

Related U.S. Application Data
(60) Provisional application No. 60/754,579, filed on Dec. 29, 2005.
(60) Provisional application No. 60/816,468, filed on Jun. 27, 2006.
(60) Provisional application No. 60/816,833, filed on Jun. 28, 2006.

(51) Int. Cl. A61M 29/02; A61M 31/00; A61M 25/098

(52) U.S. Cl. 604/103.01; 604/103.05; 604/103.1

(57) ABSTRACT

Methods and apparatus for the treatment of venous insufficiency, such as varicose veins, are described herein utilizing endovenous treatments. Such treatments may include systems to create an initial endovascular injury to the vessel wall utilizing any number of mechanisms, such as chemical, mechanical, electrical, etc. modalities. An implantable device, optionally having a sclerosing agent infused therein, may additionally be implanted along the injured tissue to promote, maintain, and otherwise enhance the tissue inflammation and scarring, thereby remodeling the diseased vessel wall.

### CROSS-REFERENCE TO RELATED APPLICATIONS

This application claims the benefit of priority to U.S. Prov. Pat. App. Ser. Nos. 60/754,579 filed Dec. 29, 2005; 60/816,468 filed Jun. 27, 2006; and 60/816,833 filed Jun. 28, 2006, each of which is incorporated herein by reference in its entirety.

### FIELD OF THE INVENTION

The present invention relates to treatment methods and apparatus for venous insufficiency. More particularly, the present invention relates to methods and apparatus for intravascularly injuring or otherwise initiating an inflammatory response along a vessel wall and propagating a continued response to ensure remodeling of the vessel wall, for example, for the treatment of varicose veins.

### BACKGROUND OF THE INVENTION

Several conventional approaches for the treatment of venous insufficiency or stasis currently exist. Examples of such treatments include surgical ligation and stripping in which damaged veins are surgically removed from the patient's body. Such a procedure requires the patient to be anesthetized and typically requires a long recovery period.

Other procedures typically include endovenous approaches for the treatment of venous reflux. There are currently several products available that provide minimally invasive endovenous treatment, of which two are commercially available and one is considered investigational. Examples include an endovenous laser which is an alternative to surgical stripping of the vein. A small laser fiber is inserted through the patient's skin, usually through a needle, and into the damaged vein. Pulses of laser light are delivered inside the vein to cause the vein to collapse and seal shut.

Other procedures include radiofrequency (RF) occlusion in which a small catheter is inserted through a needle into the skin and into the damaged vein. The catheter delivers RF energy to the vein wall, causing it to heat. As the venous tissue warms, it eventually collapses and seals shut to occlude the vessel from further blood flow.

Another example includes ultrasound guided sclerotherapy which may utilize a foam sclerosing agent. This procedure generally involves injecting a sclerosing substance (e.g., alcohol, sodium tetradecyl sulfate, etc.) in the form of a foam into the vein.

These procedures although effective, have considerable shortcomings in several areas. Both laser and RF procedures utilize high treatment temperatures to provide sufficient ablation of the venous wall. In some patients this may cause burning of the skin, requiring sedation and long recovery times. Other drawbacks generally include nerve damage, significant pain, tenderness, bruising, and skin discoloration during the post-operative period. Additionally, veins may also recanalize in time and require a second procedure. There is also considerable costs associated with the procedure as well as equipment used for these therapies. Furthermore, there are treatment restrictions imposed on these devices limiting their use for specific veins.

As for sclerotherapy utilizing foam, one difficulty associated with this procedure is an inability to control the amount of exposure that a vessel wall receives from the sclerosant. Complicating factors include diffusion and dilution of the sclerosant due to the direct injection of the foam into the blood. The result is a potential for incomplete treatment and recanalization over time. Additional complications may further include thrombo-embolic complications such as deep vein thrombosis (DVT) and pulmonary embolization as the flow of sclerosant foam is uncontrolled.

Accordingly, there exists a need for methods and apparatus which are efficacious and safe in treating patients for venous insufficiency or stasis.

### SUMMARY OF THE INVENTION

Endovenous treatments for venous insufficiency, such as varicose veins, may be accomplished by creating certain biological environments internal to the vessel being treated. Such treatments may involve initiating an inflammatory response along the tissue wall being treated to cause injury to the vessel wall and provoke a scarring response. Moreover, the creation of an initial endovascular injury to the vessel wall may be accomplished utilizing any number of mechanisms, such as chemical, mechanical, electrical, etc. modalities. An implantable device, optionally having a sclerosing agent infused therein, may additionally be implanted along the injured tissue to promote, maintain, and otherwise enhance the tissue inflammation and scarring, thereby remodeling the diseased vessel wall.

This may be accomplished by utilizing an apparatus for creating endovascular injury to tissue of a superficial, peripheral venous system, generally comprising, in one variation, an expandable outer member defining a lumen therethrough, a porous layer disposed at least partially around a surface of the outer member, and a sclerosing agent infused within the surface of the outer member. In other variations, this may also include an implantable member positioned within the lumen of the device for deployment into the inflamed tissue region.

Other examples may include an endovenous device for creating a biological environment internal to the venous system that causes obliteration and/or treatment of a diseased vein over a time period, generally comprising an occlusion member having a multi layer fiber construction which defines an internal surface and an external surface, and a plurality of fibers which originate from the internal surface such that free ends of each fiber forms the external surface of the occlusion member.

### BRIEF DESCRIPTION OF THE DRAWINGS

FIG. 1A shows an illustrative view of a great saphenous vein in the lower extremity of a patient body with a variation of the treatment system intravascularly positioned therein.

FIG. 1B shows a partial cross-sectional detail view of a variation of the catheter apparatus having an expandable outer member for temporarily contacting a vessel wall.

FIG. 2A shows a partial cross-sectional view of an outer member at least partially expanded and having an absorbent surface.

FIGS. 2B to 2D illustrate one method of absorbing a sclerosing agent into the outer member by soaking the outer member within a sheath filled with the agent.

FIGS. 3A and 3B illustrate a method of positioning a catheter apparatus within a vessel and exposing the outer member by removing a constraining sheath.

FIG. 4A illustrates a method of positioning and expanding a catheter apparatus by inflating the outer member directly into contact with the vessel walls.

FIGS. 4B and 4C illustrate the inflammatory response and remodeled vein after cellular matrix formation.

FIG. 5 shows another variation of an expandable outer member made from a shape memory alloy or polymer constrained within a sheath member.

FIGS. 6A and 6B illustrate a method of positioning and expanding the catheter variation shown in FIG. 5 in which the sheath member is removed allowing the shape memory alloy or polymer to expand the sclerosing agent into contact against the vessel walls.

FIG. 7A illustrates another side view of a treatment system positioned within a great saphenous vein.

FIG. 7B is a partial cross-sectional detail perspective view illustrating a variation of the treatment system having a catheter, a guidewire, and an implantable member positioned within a vein to be treated.

FIG. 8A is partial cross-sectional side view of another variation of the catheter device configured to hold an implantable member therein and further having an outer porous coating capable of retaining and releasing a sclerosing agent in a controlled manner into or against the vessel walls.

FIGS. 8B and 8C are partial cross-sectional detail side views of variations of the treatment system distal end both with and without an implantable member contained within, respectively.

FIGS. 9A to 9F illustrate another method of applying a sclerosing agent into or upon the outer member of a catheter device having an implantable member contained therein where a user can optionally apply any particular sclerosing agent to the outer member prior to inserting the catheter into a diseased vessel.

FIGS. 10A and 10B illustrate side views of a catheter device carrying an implantable member therein and having an expandable outer member soaked with a sclerosing agent while sheathed during delivery and with the sheath removed for deploying the expandable outer member into contact against the vessel walls.

FIGS. 11A to 11C illustrate side views of one method for delivering and deploying an implantable member; as shown, the implantable member is deployed while one or more mechanical arms induce trauma to the vessel walls while unsheathing the implantable member and further after or while applying the sclerosing agent to the vessel walls via the expandable outer member.

FIGS. 12A and 12B illustrate another method for treating the vessel walls by initially positioning and unsheathing an expandable outer member into contact against the vessel walls to expose the tissue to the sclerosing agent infused into the outer member.

FIGS. 13A and 13B further illustrate the unsheathed outer member of FIGS. 12A and 12B expanded into contact against the vessel walls and inducing chemical trauma thereto via the sclerosing agent infused within the outer member to induce an inflammatory response.

FIGS. 14A to 14C further illustrate the traumatized vessel walls from FIGS. 13A and 13B with the sclerosing catheter removed and an implant delivery catheter inserted in its place in which mechanical trauma is further induced against the vessel walls prior to or while the implantable member is deployed into the vessel and into contact against the vessel walls to further induce additional trauma thereto.

FIGS. 14D to 14F illustrate exemplary changes in the biodegradable implant as well as an example of a step-by-step process of vein tissue remodeling resulting from exposure to the sclerosant-eluting degrading implant.

FIG. 15A shows a partial cross-sectional side view of a catheter system having an implantable member disposed and having one or more radially expandable members for mechanically inducing trauma to the vessel walls.

FIGS. 15B and 15C illustrates yet another variation of a catheter having a slotted tubular sheath restrained by a constraining member which is actuatable via a tensioning member such as a pullwire.

FIGS. 16A and 16B illustrate another method of initiating trauma to the vessel wall via thermal or electrical energy.

FIGS. 16C and 16D illustrate another method of initiating trauma to the vessel wall while simultaneously deploying an implantable member.

FIGS. 16E to 16H illustrate yet another method of initiating trauma to the vessel wall and subsequently deploying an implantable member into the treated vessel.

FIG. 17A illustrates a side view of one variation of an implantable member.

FIG. 17B is a partial cross-sectional side view of an implantable member having a guidewire routed through a perforated delivery catheter.

FIGS. 18A to 18F illustrate design variations of the implantable member.

FIGS. 19A and 19B illustrate another method for applying or infusing the biodegradable implantable member with a sclerosing agent of a choice prior to insertion within a patient.

FIG. 19C shows a partial cross-sectional view of a segment of the implantable member positioned within a perforated delivery catheter after application of a sclerosing agent.

FIGS. 20A to 20C illustrate another method for applying or infusing a sclerosing agent onto the implantable member prior to insertion within the delivery catheter, e.g., during its manufacturing process.

FIGS. 21A to 21D illustrate a method for deploying a sclerosant agent-eluting implantable member within a vein to further enhance an inflammatory response from the vessel walls.

FIGS. 22A and 22B illustrate detail cross-sectional views of a deployed implantable member and the resulting inflammatory cellular response.

FIG. 23A illustrates a biodegradation process of the implantable element accompanied by sclerosing agent release.

FIGS. 23B to 23F illustrate exemplary changes in the biodegradable implant as well as an example of a step-by-step process of vein tissue remodeling resulting from exposure to the sclerosant-eluting degrading implant.

FIG. 24A shows an example of another variation for an implantable member having a spherical or cylindrical configuration with one or more fibers depending therefrom.

FIG. 24B shows another variation of the implant of FIG. 24A illustrating multiple cylindrical or spherical members connected to one another for deployment into a vessel.

FIGS. 25A and 25B show combinations of a helical member having loops or strands protruding therefrom for implantation into the vessel.

FIG. 26 shows another variation comprising of an elongated tubular member having loops or strands protruding therefrom.

FIG. 27 illustrates one method of deploying one or more cylindrical or spherical implantable members from a delivery catheter into the vessel.

FIG. 28 illustrates another method where a deployment sheath of the delivery catheter defines an abrasive outer surface for inducing endothelial damage to the vessel wall.

FIG. 29 shows a variation on the delivery catheter which utilizes one or more deployable loops or other mechanisms for inducing an inflammatory response along the tissue wall.

FIG. 30 shows another variation of an implantable member where the member is a tubular shape having multiple fibers depending therefrom.

### DETAILED DESCRIPTION OF THE INVENTION

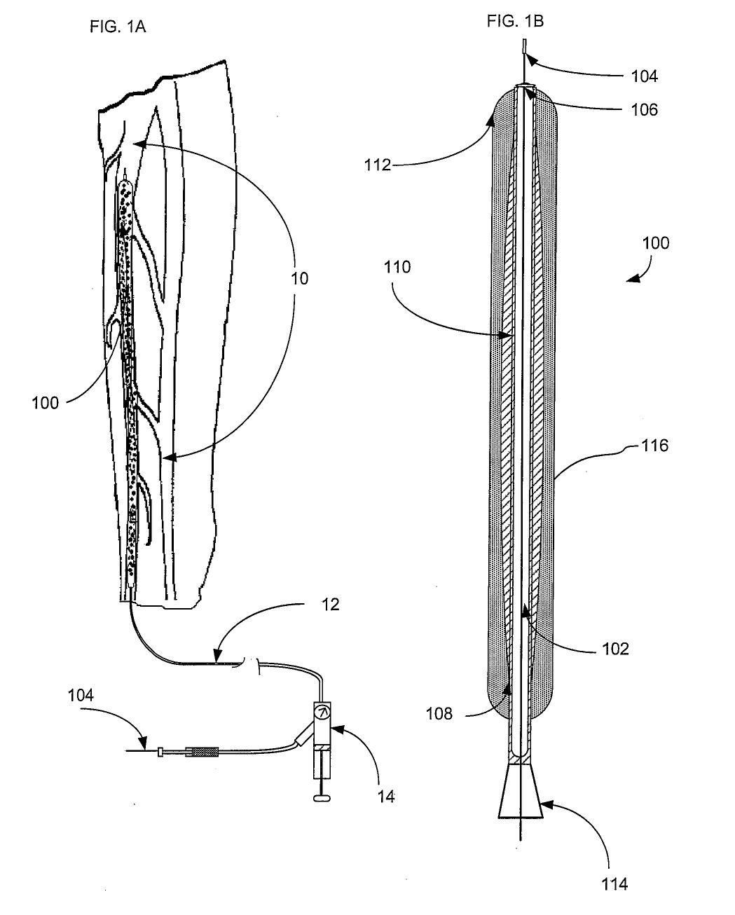

FIG. 1A illustrates an illustrative view of a great saphenous vein 10 in the lower extremity of a patient body with one variation of the catheter treatment system 100 intravascularly positioned therein. The catheter treatment system 100 may be introduced into the patient body via percutaneous access through the patient's skin and into the saphenous vein 10 to be treated at a location distal to the diseased region. The catheter system 100 may be advanced intravascularly through the vein 10 and proximal of the sapheno-femoral junction until the portion to be treated has been reached and/or traversed by the catheter system 10. As further shown, the catheter treatment system 100 may be connected via an inflation/deflation tubular member 12 to a pump 14 positioned externally of the patient.

Moreover, one or more access ports may be incorporated with the system to allow for access by other devices, such as guidewire 104, which may be optionally advanced distally of the catheter system 100 to facilitate access through the vasculature. Additionally, a proximal portion 114 of the catheter assembly 100 may further define a flared or tapered portion to facilitate the insertion and access of a guidewire 104 into and through the assembly 100.

FIG. 1B illustrate one variation of an elongated tubular catheter assembly 100, having a distal and a proximal end and a lumen 102 to optionally receive a guidewire 104 therethrough. An echogenic or radio-opaque marker 106 may be optionally disposed near or at a distal end of the catheter to facilitate visualization and positioning of the device within a vessel 10 via, e.g., ultrasound, fluoroscopy, etc. Aside from an echogenic or radio-opaque marker 106, an illuminating member such as a light emitting diode, chemiluminescent marker, etc., may be positioned upon the catheter and illuminated during advancement and placement within the vessel. The illuminated marker may be sufficiently bright enough to illuminate through the vessel and skin of the patient to allow for the surgeon or user to track a location of the catheter via direct visualization without utilizing other imaging modalities.

The catheter assembly 100 may also include an expandable outer member 108, such as an inflation balloon, and an inflation lumen 110 that is in fluid communication with the outer member 108. The outer surface 116 of the outer member 108 may be completely or at least partially covered with a highly absorbent and/or porous material such as foam 112. The outer surface 116 of the outer member 108 may be comprised of a porous material to facilitate the absorption and retention of a sclerosing agent therein. Once the catheter system 100 has been advanced and desirably positioned within the vessel to be treated, the sclerosant contained within the outer surface 116 may be applied to or against the interior of the vessel wall to be treated, as further described below.

Although a single expandable member 108 is illustrated, one or more expandable members positioned in series relative to one another may alternatively be utilized. Each of the expandable members may be connected via a common inflation and/or deflation lumen to expand each of the expandable members. Alternatively, each of the expandable members may be connected via its own inflation/deflation lumen such that individual balloons may be optionally inflated or deflated to treat various regions of the vessel. Moreover, the expandable member 108 may be comprised of an inner balloon member and an outer balloon member, where each inner and outer balloon member is configured to have varying or different elasticity and compliance rates.

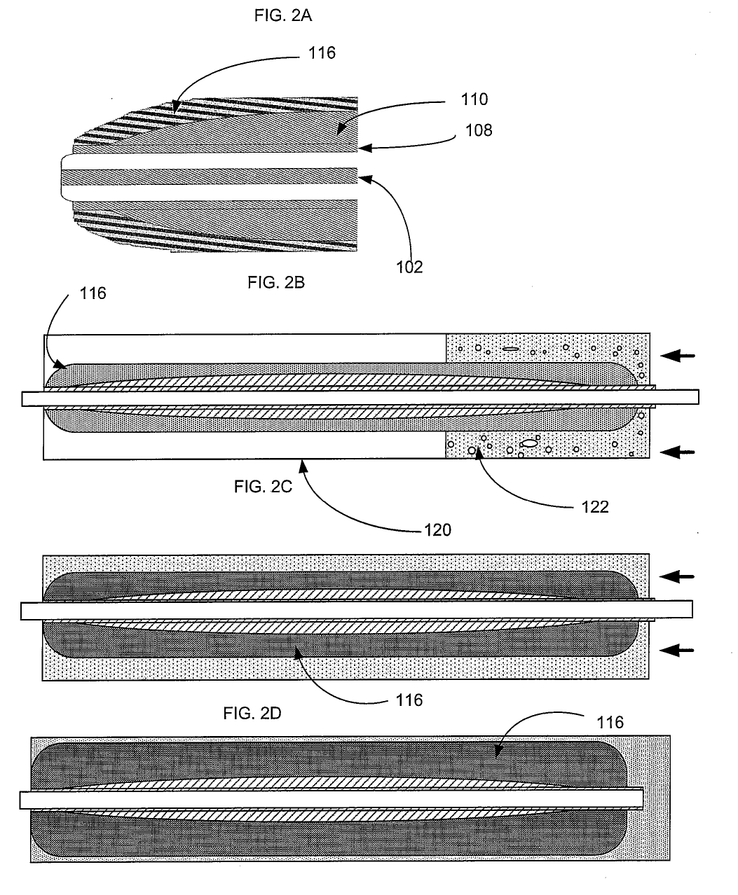

FIG. 2A shows a partial cross-sectional view of the distal segment of the outer member 108 at least partially expanded and having absorbent surface 116 containing a sclerosing agent absorbed therein.

FIG. 2B illustrates one method for applying a sclerosing agent into or upon the outer member 116. An outer sheath 120, such as tubular sheath, may be positioned around at least a portion of the outer surface. Once outer sheath 120 has been desirably positioned, a sclerosing agent 122 may be injected or poured into sheath 120 such that the outer surface 116 is immersed at least partially within agent 122 and eventually takes up or absorbs agent 122 via the porous outer surface 116, as shown by the absorption of sclerosant in FIGS. 2C and 2D.

The sclerosing agent utilized may comprise any number of agents. For example, some agents which may be used may include, but are not limited to: alcohol, ethanol, chemotherapeutic agents, cytostatic agents, cytotoxic agents, sodium tetradecyl sulfate, Doxycycline, OK-432, saline and aethoxysclerol solutions, etc., and combinations thereof.

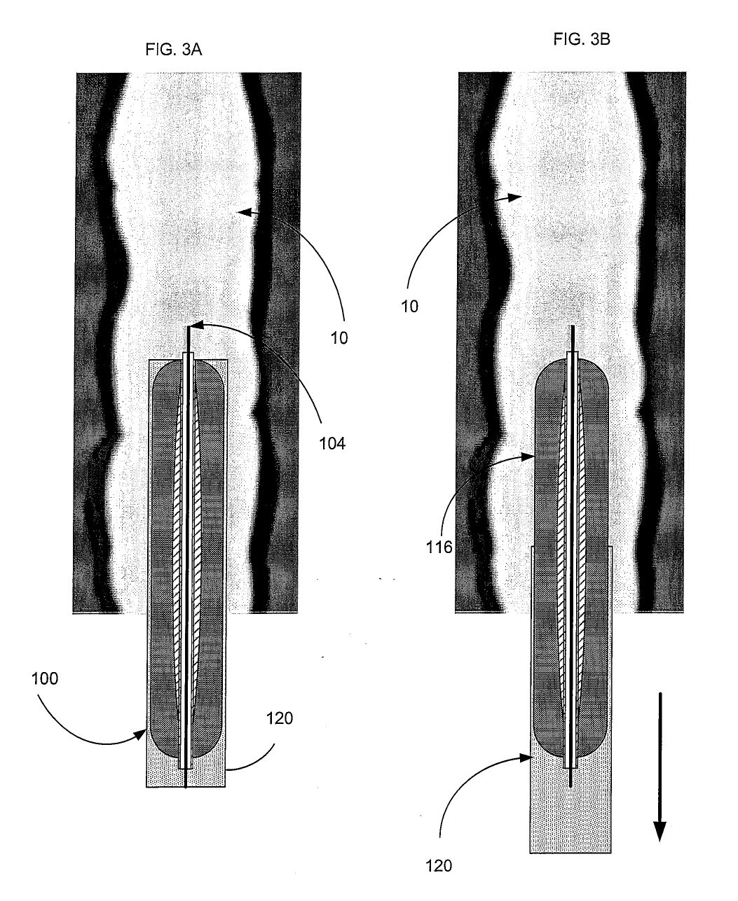

Once the outer surface 116 has absorbed a desirable amount the sclerosing agent 122, the catheter assembly 100 (optionally with outer sheath 120) may be introduced into the vasculature and advanced to the tissue location to be treated, as shown in FIG. 3A. Once desirably positioned adjacent to or proximate to the vessel wall 10 to be treated, tubular sheath 120 may be pulled proximally or outer surface 116 may be advanced distally relative to sheath 120 such that the outer surface 116 is exposed within the vessel, as shown in FIG. 3B.

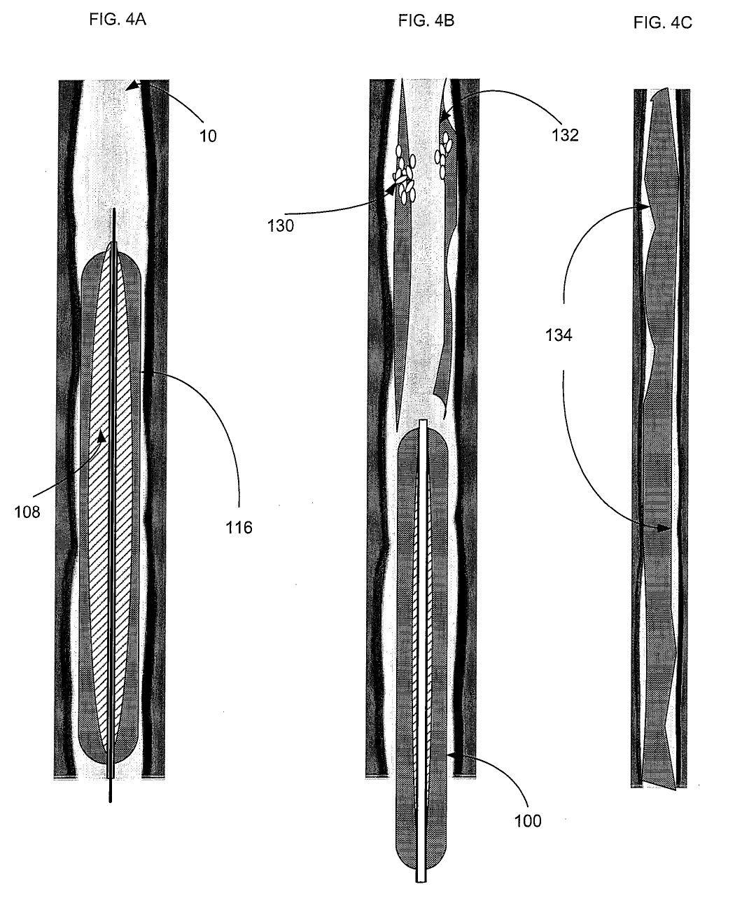

Once exposed, catheter assembly 100 may be manipulated to contact the vessel walls to be agitated. Alternatively, expandable outer member 108 may be inflated via pump 14 through inflation/deflation tube 12 to inflate expandable outer member 108 and appose its porous surface 116 uniformly or otherwise against the interior wall of the vein 10, as shown in FIG. 4A. Pressure from the catheter outer layer 116 may facilitate the application or delivery of the sclerosing agent 122 that is contained in the porous surface 116 directly, uniformly, and efficiently to the vessel wall with minimum dilution and diffusion. The desired length and diameter of the exposed vessel may then injured 132 thus evoking an inflammatory cellular response 130. Once the desired sclerosant has been applied for a desired period of time, as shown in FIG. 4B, the catheter system 100 may be deflated and removed from the vessel or to another region to be treated. FIG. 4C illustrates the resulting remodeled vein 134 after complete cellular matrix formation.

FIG. 5 shows another variation of an expandable outer member 108 made from a shape memory alloy (such as a Nickel-Titanium alloy) or shape memory polymer or combination thereof constrained within sheath member 120. The outer member 108 may be collapsed and confined in its low-profile shape by constraining it inside sheath member 120.

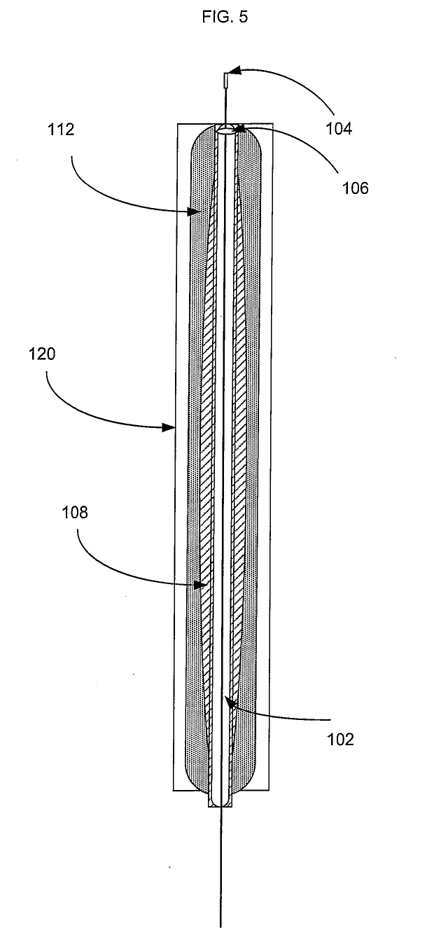

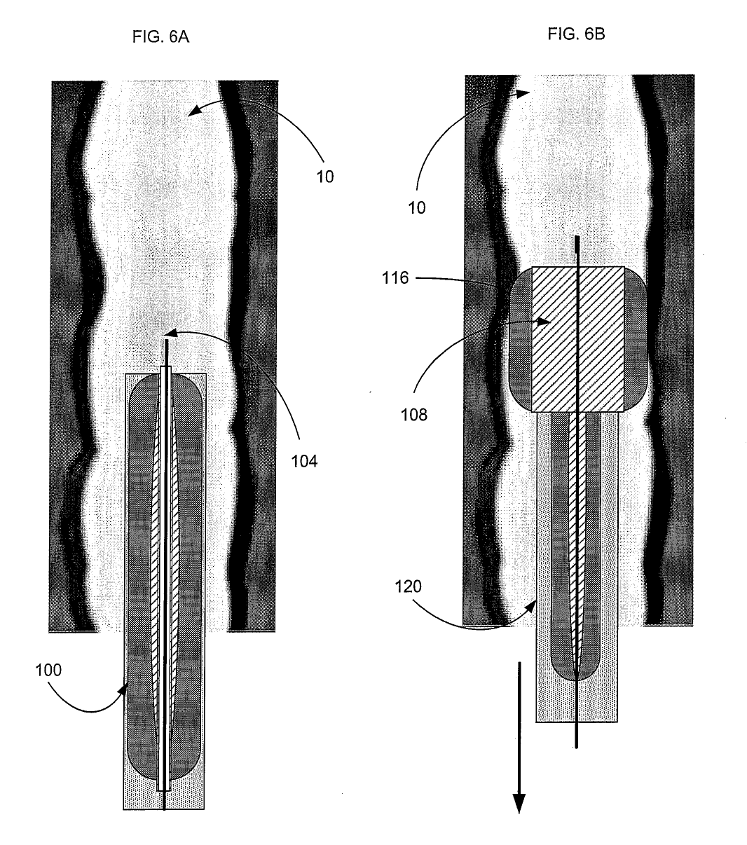

As shown in use in FIG. 6A, the device may be advanced over guidewire 104 and placed in a desired location within the vessel 10. As shown in FIG. 6B, sheath member 120 may then be pulled proximally or outer member 108 may be advanced distally relative to sheath 120 leaving outer member 108 to expand and expose its surface 116 for contact against the vessel wall, as above. Once the sclerosant has been applied for the desired period of time, the sheath 120 may be advanced distally over outer member 108 or outer member 108 may be drawn proximally within sheath 120 to collapse the device for removal from the vessel.

Aside from the variations described utilizing the application of a sclerosing agent directly to the vessel walls to be treated, additional variations may further provide for a system to intravascularly treat venous insufficiency by induction and facilitation of a controlled tissue remodeling process leading to a scar tissue formation and obliteration of the diseased vessel in combination with a biodegradable and/or bioresorbable implantable device. Such an implantable device may include, but is not limited to, polymer-based biodegradable and/or bioresorbable implantable devices.

Additionally, methods for trauma induction to the inner surface of the vessel coupled with delivery and implantation of the implantable device, methods of making delivery and implantable devices, and methods of treatment that utilize these devices are also described herein.

A large number of different types of materials which are known in the art may be utilized in the implantable device to be inserted within the body and later dissipated. Such bioabsorbable and/or biodegradable materials utilized in the implantable devices may be adapted to dissipate upon implantation within a body, independent of which mechanisms by which dissipation can occur, such as dissolution, degradation, absorption, and/or excretion. The actual choice of which type of materials to use may readily be made by one ordinarily skilled in the art. The terms bioresorption and bioabsorption and/or biodegradation can be used interchangeably and refer to the ability of the polymer or its degradation products to be removed by biological events, such as by fluid transport away from the site of implantation or by enzymatic activity or by cellular activity (e.g., phagocytosis). Accordingly, both bioabsorbable and biodegradable terms will be used in the following description to encompass absorbable, bioabsorbable, and biodegradable, without implying the exclusion of the other classes of materials.

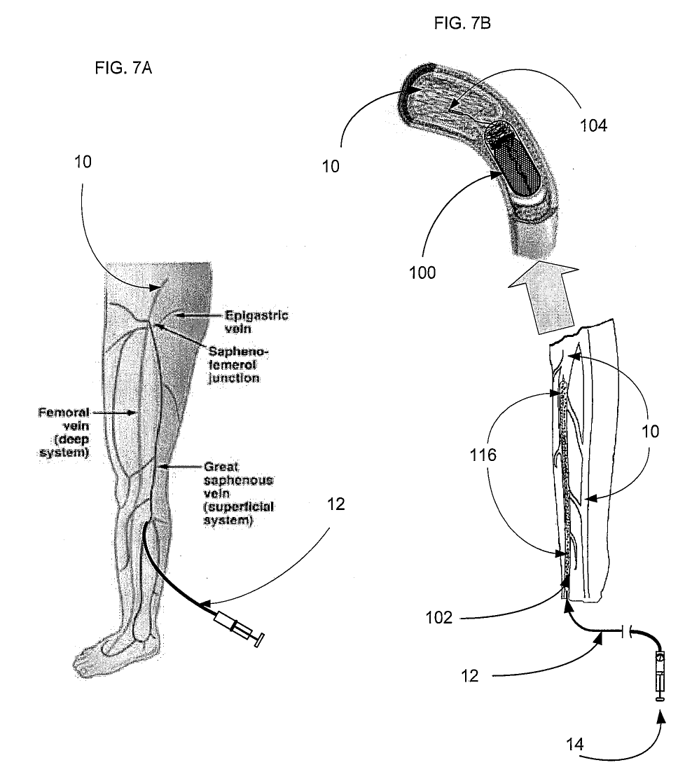

FIGS. 7A and 7B illustrate a variation of an elongated and tubular catheter system 100 positioned within the saphenous vein 10 as previously shown and described above. FIG. 7B illustrates a partial cross-sectional detail view of the catheter system 100 advanced over a guidewire 104 within the vessel lumen. Also shown is a variation of the catheter system 100 connected via tubular member 12 to a pump or syringe 14 located externally of the patient body with the absorbable outer surface 116 positioned within the vessel 10.

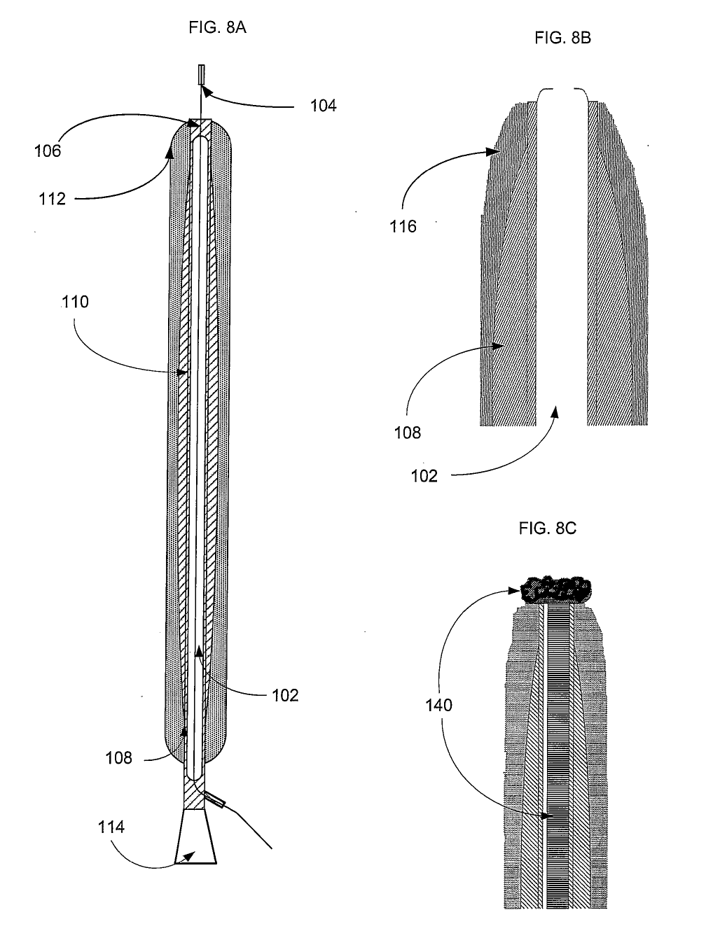

Also as described above, the catheter in FIG. 8A shows a partial cross-sectional view of the expandable outer member 108 surrounded by the absorbable outer foam surface 112. FIG. 8B illustrates a detail cross-sectional view of the expandable outer member 108 and the surrounding outer surface 116 defining a lumen 102 therethrough. FIG. 8C shows one variation of the device having a bioabsorbable polymeric implantable device 140 positioned within the lumen 102 of the catheter device. The implantable device 140 is described in further detail below.

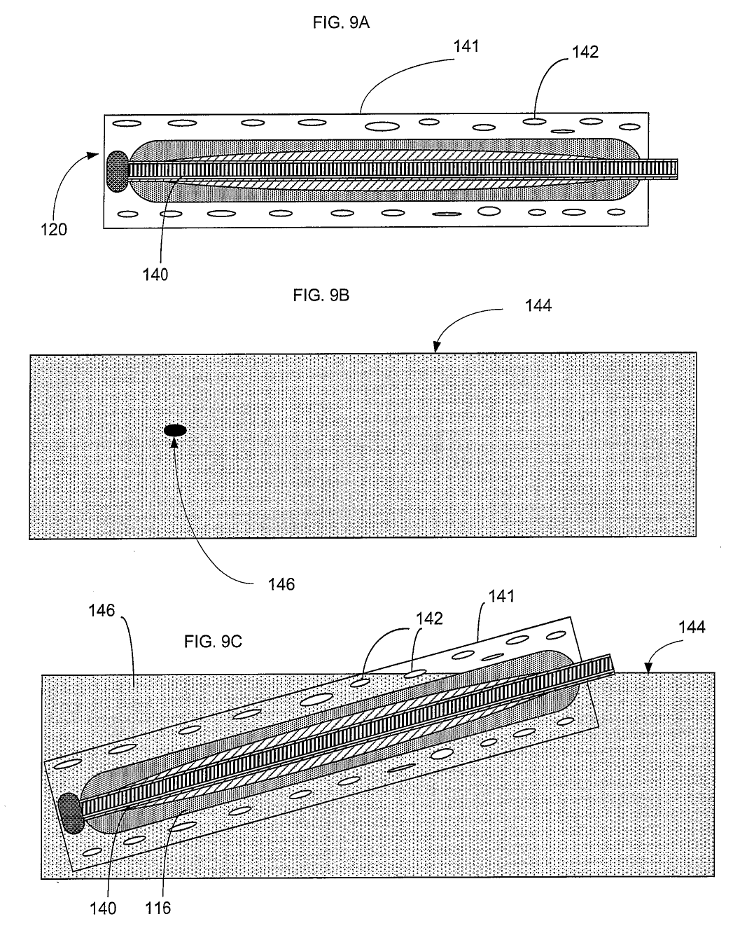

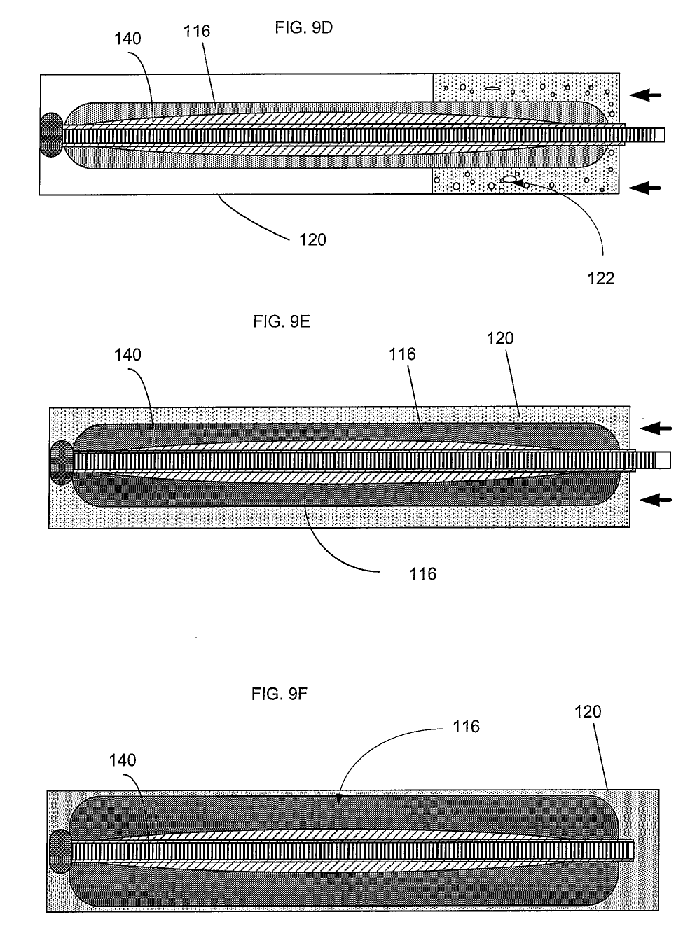

In preparing the catheter assembly and bioabsorbable implantation device for use in a patient body, one method is illustrated in FIGS. 9A to 9C. As illustrated, the catheter device having the implantable member 140 positioned within may be placed within a tubular sheath 120, as previously described, or within a perforated sheath 141 which defines a plurality of openings or holes 142 over its surface. The assembly of the catheter and implant device may be immersed entirely or partially within a sclerosant bath 144 containing a sclerosing agent 146, as shown in FIG. 9B. As the catheter and implantable device 140 is immersed within the bath 144, the sclerosing agent 146 may flow through the openings 142 into the perforated sheath 141 to become absorbed by the outer surface 116 as well as by the implantable member 140. Once the assembly has absorbed a desirable amount of sclerosant, the assembly may be advanced into the patient body, as described in detail below.

FIGS. 9D to 9F illustrates another method for applying a sclerosing agent to the porous surface 116 of the catheter by direct injection into the outer sheath 120 or by immersion. As described above, the sclerosant 122 may be injected into the sheath 120 to be absorbed by outer surface 116 and by the implantable device 140, as illustrated in FIGS. 9E and 9F.

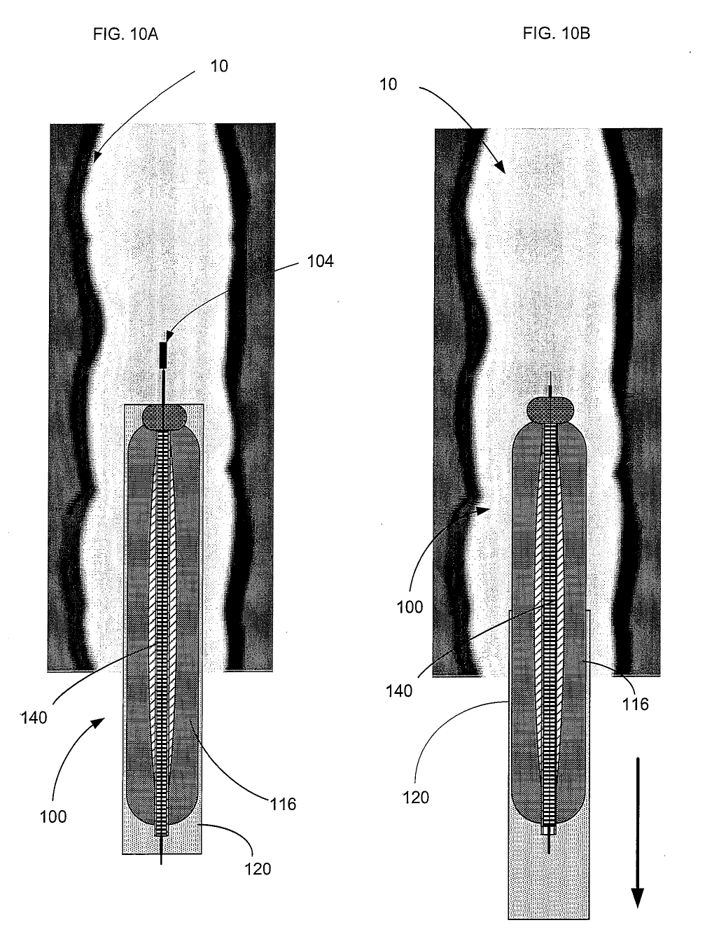

In use, as shown in FIGS. 10A and 10B, the catheter system 100 having the sclerosant-loaded outer surface 116 may be advanced into the vessel 10 to be treated over a guidewire 104 to the treatment site under ultrasound guidance or through other imaging methods. Once desirable positioned, the tubular sheath 120 may be pulled proximally or the catheter may be advanced distally relative to the sheath 120, thereby exposing the porous outer surface 116 that is loaded with the sclerosing agent for contact against the interior of the vein 10, as shown in FIG. 10B.

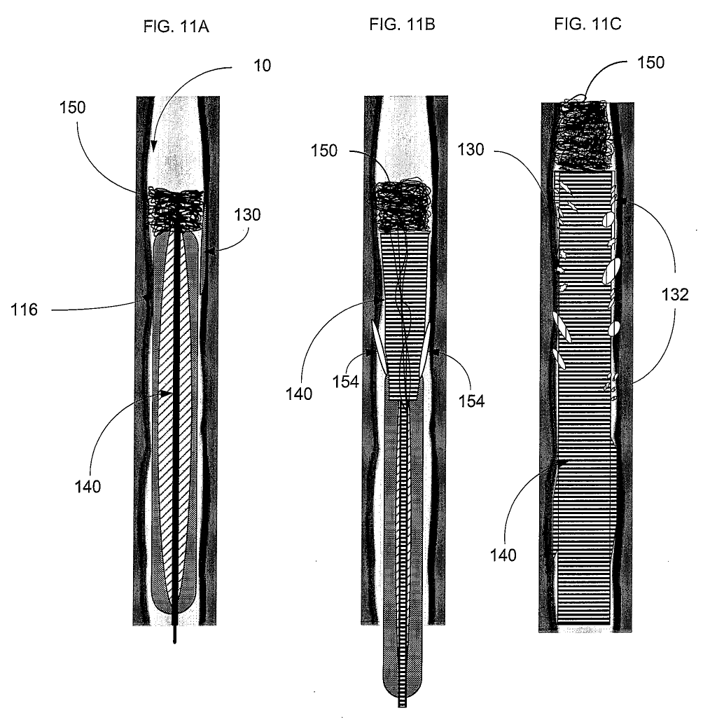

FIGS. 11A to 11C illustrate the application of the sclerosant to the vessel walls as well one method for deploying the implantable device 140. Once the catheter has been desirable positioned within the vessel to be treated, the outer surface 116 may be expanded into contact against the tissue walls by application of an inflation pressure bringing the outer coated surface 116 into full contact with the vessel wall.

One or more deployable mechanical arms or members 154 may be deployed from the catheter to contact against the vessel walls to further induce an inflammatory response as the members 154 are pulled proximally along the tissue wall. Meanwhile, the implantable member 140, in this variation having a fibrous distal end 150, may be ejected from the lumen of the catheter into the vessel, as shown in FIG. 11B. Inflammation 130 activated by the mechanical trauma from members 154 coupled with application of the sclerosing agent may trigger an acute cellular response 132, which is maintained by deployment and implantation of the implantable device within the vessel, as shown in FIG. 11C. The implantable device 140 remains within the vessel in contact with the tissue wall to continue and maintain the cellular response until the device is finally absorbed and/or degraded, leaving behind the remodeled tissue wall, as described in further detail below.

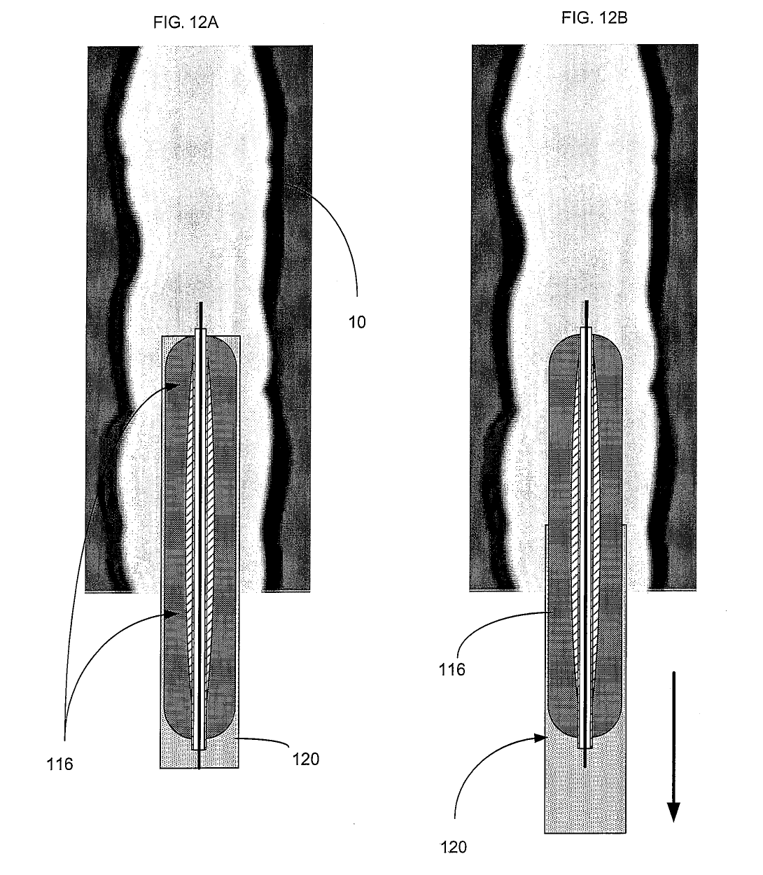

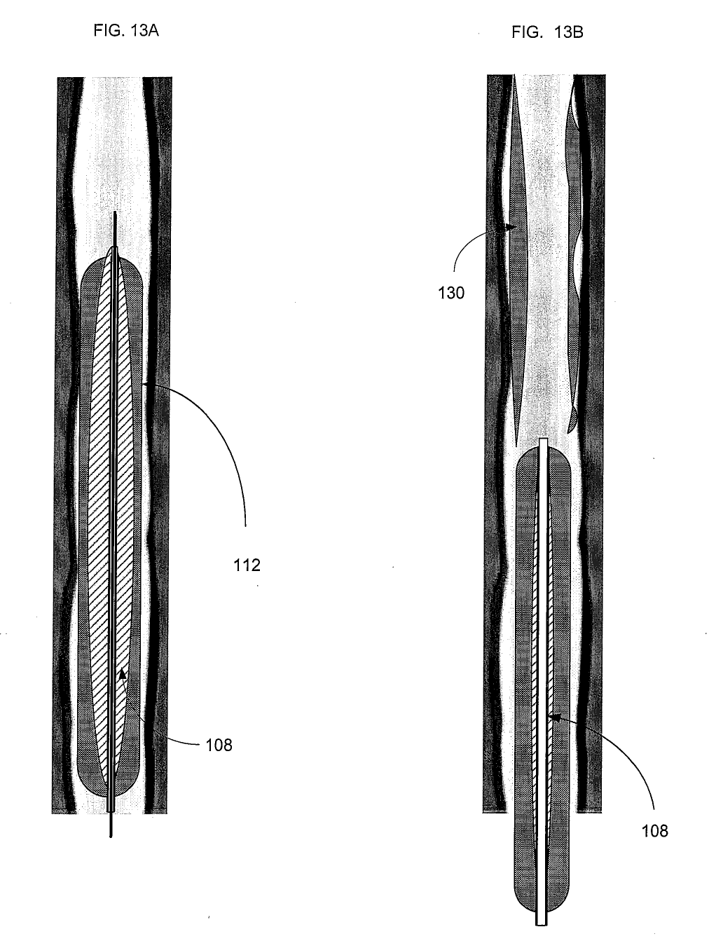

In another method for initiating an inflammatory response and for implanting an absorbable device, a multiple-step method is shown where in FIGS. 12A and 12B, a catheter device constrained by sheath 120 and having a sclerosant-laden outer surface 116 may be advanced into the vessel and unsheathed, as described above. Once unsheathed, the expandable member 108 may be inflated or otherwise expanded to present the surface 116 into contact against the tissue wall, as shown in FIG. 13A. Once an inflammatory response 130 has been initiated, as shown in FIG. 13B, the catheter may be deflated and removed from the vessel.

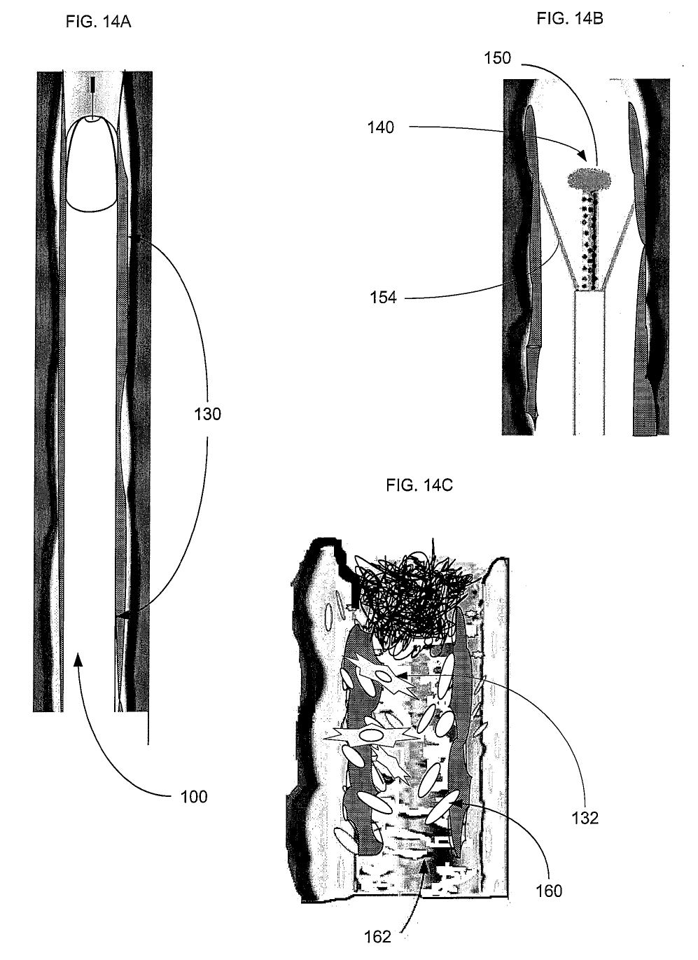

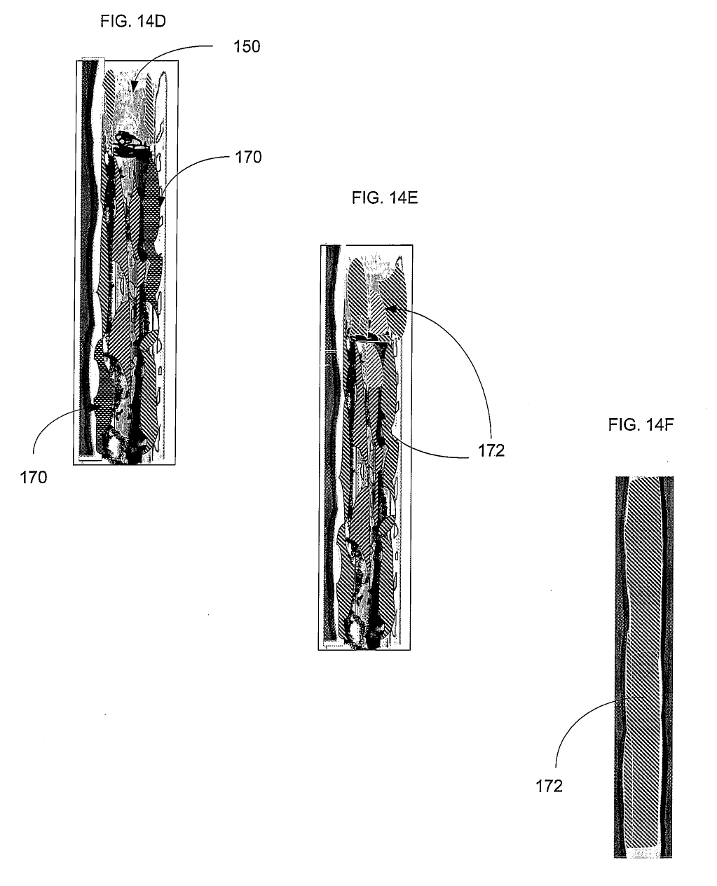

A second catheter device may be advanced into vessel and positioned adjacent the inflamed tissue 130, as shown in FIG. 14A. With the sclerosing catheter removed and an implant delivery catheter inserted in its place, further mechanical trauma may be induced against the vessel walls by members 154 prior to or while the implantable member 140 is deployed into the vessel and expanded into contact against the vessel walls to further induce additional trauma thereto, as shown in FIG. 14B. With the implantable device expanded 162 against the tissue wall, the inflammation activity of the tissue, as further activated by the mechanical trauma and coupled with sclerosing agent progresses into a chronic cellular response 160, as shown in FIG. 14C.

FIGS. 14D to 14F illustrate exemplary changes in the biodegradable implant as well as an example of a step-by-step process of vein tissue remodeling resulting from exposure to the sclerosant-eluting degrading implant. As the inflammation continues, granulation tissue formation 172 occurs, as shown in FIG. 14E, eventually leading to scar tissue formation 172 and remodeling, as shown in FIG. 14F.

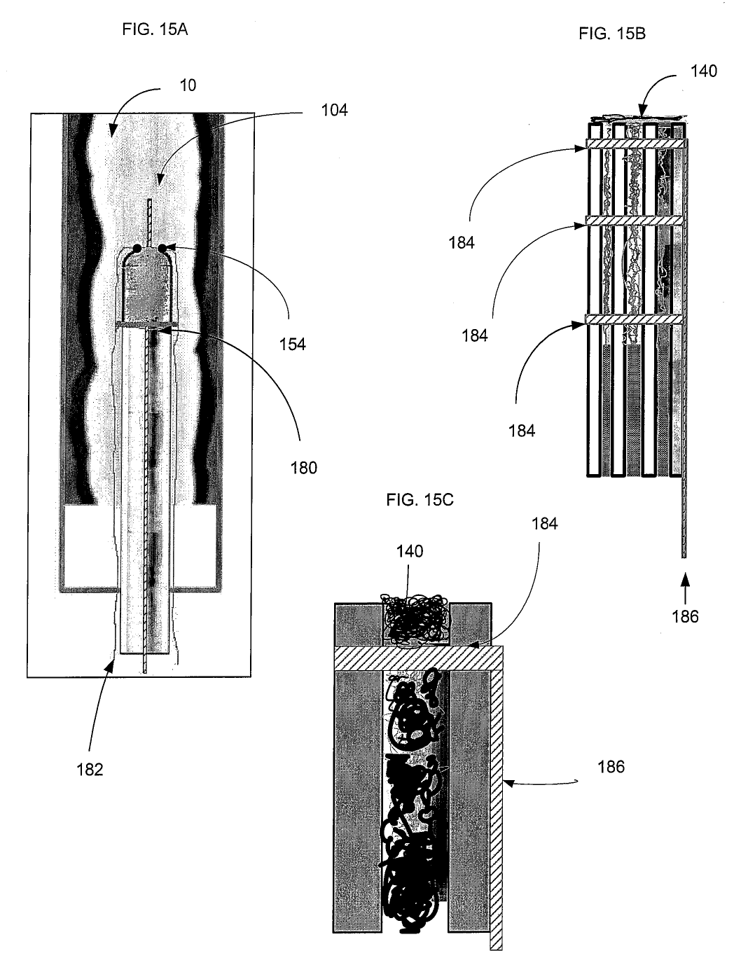

In another variation of the catheter assembly, as illustrated in FIG. 15A, the catheter assembly may include one or several deflectable members 154 for the induction of mechanical injury to the interior of the vein 10, as described above. These deflectable members 154 may surround the implantable member contained within the catheter and may be activated by any number of mechanisms, such as by the tensioning of the draw string or pullwire 182 and deflection against a fixed segment 180.

In yet another variation shown in FIGS. 15B and 15C, a distal segment of the catheter assembly containing the bioresorbable polymeric implant 140 may be configured as a slotted tubular sheath with several restricting segments 184 which may be attached to a draw string or pullwire 186. The restricting segments 184 may be configured to confine the slotted distal section and the bioabsorbable implant 140. Upon tensioning or pulling of the draw string or pullwire 186, the restricting segments 184 may be moved to release the slotted tubular sheath causing its deflection against interior of the vein 10 to induce mechanical injury.

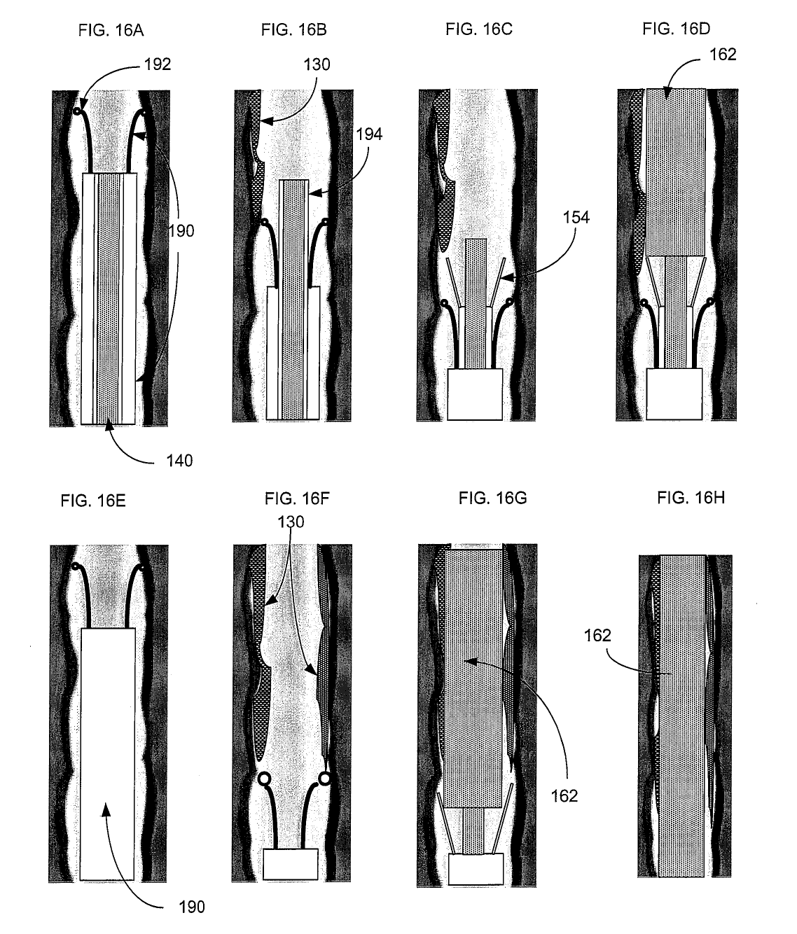

In yet another variation as illustrated in FIGS. 16A and 16B, the interior of the vein 10 may be exposed to thermal energy to invoke an inflammatory response. The catheter system 190 may have one or more electrodes configured as extendable arms or members 192 to contact the tissue walls. The applied energy may alternatively be in the form of cryogenic, RF, laser energy, etc., or combinations thereof. Moreover, the catheter may further define a lumen within which a bioabsorbable implant 140 is encased for deployment immediately after the application of the energy. FIGS. 16C and 16D illustrate the simultaneous exposure of the vein 10 to the applied energy and release of bioabsorbable implant 140, which in turn will assume its expanded state 162. The release of the implant 140 may be facilitated by the separation of mechanical deflectors 154.

FIGS. 16E to 16H illustrate another method of treatment and implant deployment where exposure of the vein 10 to applied energy, thermal or otherwise, may be a first step of the treatment, causing an inflammatory response 130, as shown in FIGS. 16E and 16F. Following the application of the energy to the tissue wall, the bioabsorbable implant 140 may be deployed subsequently to assume its expanded position 162 upon implantation, as shown in FIGS. 16G and 16H.

Now turning to examples of the bioabsorbable polymeric materials which may be utilized with the implantable device. As mentioned above, the implantable device can comprise a bioabsorbable material. Such materials may be selected from any number of bioabsorbable homopolymers, copolymers, or blends of bioabsorbable polymers. In some variations, an implantable device architecture can comprise a synthetic biocompatible, bioabsorbable polymer or copolymer, a natural biocompatible, bioabsorbable polymer or copolymer or combinations thereof.

Several synthetic bioabsorbable, biocompatible polymers have been developed for use in medical devices. These widely used materials include polyglycolic acid (PGA), polylactic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS). In general, these materials biodegrade in vivo in a matter of months, although some more crystalline forms can biodegrade more slowly. These materials have been used in orthopedic applications, wound healing, interventional cardiology and radiology applications, and extensively in sutures after processing into fibers.

A number of natural biodegradable polymers can also be used for the constriction of the parts and components of the implantable element, including but not limited to: fibrin, fibrinogen, elastin, collagens, gelatin, cellulose, chitosan, extracellular matrix (ECM), carrageenan, chondroitin, pectin, alginate, alginic acid, albumin, dextrin, dextrans, gelatins, mannitol, n-halamine, polysaccharides, poly-1,4-glucans, starch, hydroxyethyl starch (HES), dialdehyde starch, glycogen, amylase, hydroxyethyl amylase, amylopectin, glucoso-glycans, fatty acids (and esters thereof), hyaluronic acid, protamine, polyaspartic acid, polyglutamic acid, D-mannuronic acid, L-guluronic acid, zein and other prolamines, alginic acid, guar gum, and phosphorylcholine, as well as co-polymers and derivatives thereof.

Various cross linked polymer hydrogels can also be used in constructing core or coating components of the implant.

One of the implant variations may have a biodegradation rate where the distal segment of the implantable element has a slower biodegradation rate than the proximal segment to further protect against the release and migration of debris into the femoral vein. There are a variety of cross-linking methods utilizing chemical, physical and combined technologies to achieve a desirable biodegradation rates.

In an additional variation where one of the components is made of collagen, a cross-linking density may be controlled through the addition of a selected amount of a bi-functional reagent to the collagen. The bi-functional reagent may include an aldehyde and/or a cyanamide. The aldehyde may include a glutaraldehyde, for example. The core and the outer coating of the implant may include collagen and a cross-linking density of the first and second portions may be different and to be controlled by an application of energy to the collagen. The application of energy may include dehydrothermal processing and/or exposure to UV light or radiation, for example. The various components of the implant may include collagen and a cross-linking density of the first and/or second components may be controlled by a combination of dehydrothermal processing and exposure to cyanamide.

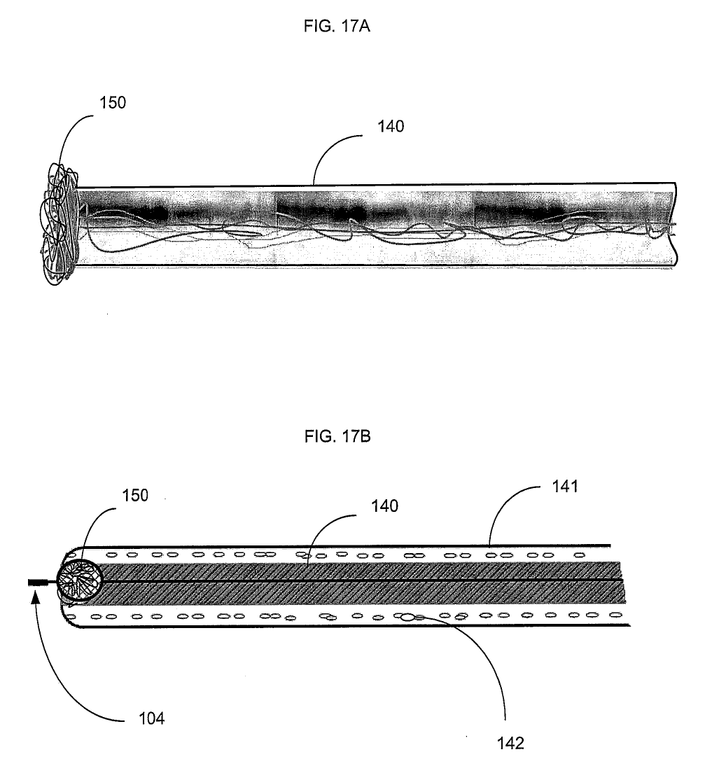

FIGS. 17A and 17B illustrate some examples of various configurations of the biodegradable implant 140 of the varicose vein treatment system. The implantable member 140 may comprise, e.g., collagenous-based biomaterials, exposed fibrous mesh component, and core fibrous mesh component and various outer layer coating components. The example shown in FIG. 17A illustrates an unconstrained implant 140 having an optional fibrous mesh component 150 at a distal end of the implant 140. Fibrous component 150 may be exposed upon implantation and further act to not only maintain the inflammatory response and temporary in-growth of the tissue, but it may also act as a filtering mechanism allowing blood flow to continue unimpeded through the fibrous component 150 but also capturing any errant thrombus, errant debris, or other materials within the vessel. FIG. 17B illustrates another example of an implantable member 140 constrained within a sheath 141 prior to deployment within the vessel.

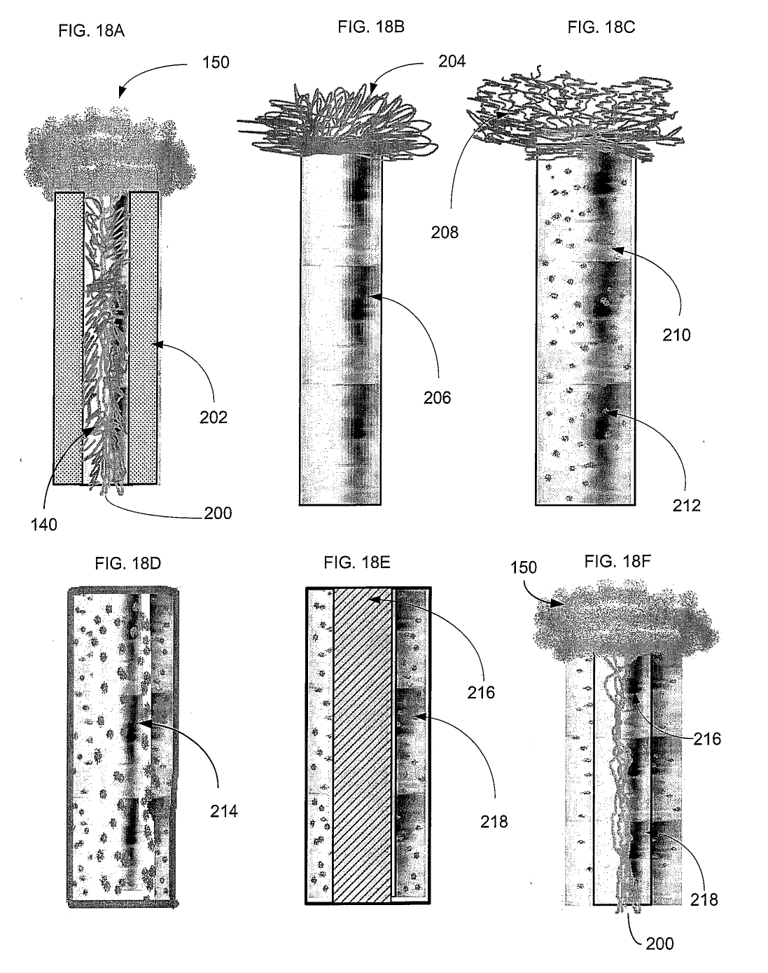

FIG. 18A depicts a longitudinal cross-sectional view respectively of another example of a biodegradable implant 140 having an exposed fibrous mesh component 150 made out of an entangled network of PGA-PLA fibers, core fibrous mesh component 200 made out of one or more strings of PGA-PLA coupled with the fibrous mesh component 150 and porous sleeve 202 made of collagen sponge.

FIG. 18B depicts a longitudinal view of another variation of a biodegradable implant having an exposed fibrous mesh component 204 made out of looped network of PGA-PLA fibers and rolled into a non porous film 206 made of collagen.

FIG. 18C depicts a longitudinal view of another variation of a biodegradable implant having an exposed fibrous mesh component 208 made out of entangled, looped or knitted network of PGA-PLA fibers, rolled into a porous matrix 210 made of collagen sponge with a designed pore architecture 212.

FIG. 18D depicts a longitudinal view of another variation of a biodegradable implant having a cylindrical porous matrix 214 made of collagen with a designed pore architecture.

FIG. 18E depicts a cross-sectional longitudinal view of another variation of a biodegradable implant having an inner non-porous component 216 made out of any non-porous biodegradable biomaterial (in this particular variation also collagen) and outer porous sleeve 218 made of collagen sponge with a designed pore architecture.

FIG. 18F depicts a longitudinal cross-sectional view of another variation of a biodegradable implant having an exposed fibrous mesh component 150 made out of entangled network of PGA-PLA fibers, core fibrous mesh component 200 made out of long string of PGA-PLA coupled with 150, incorporated into an inner non-porous component 216 and rolled into a porous matrix 218 made of collagen sponge.

Moreover, any of the implants described herein may optionally comprise a non-degrading proximal or distal portion made, e.g., from a non-absorbable polymeric or metallic material. Such a non-absorbable segment may be made from various materials, such as polyester fibers, ePTFE, PTFE, Platinum, Gold, stainless steel, Nickel-Titanium alloys, and combinations thereof, in forms of wire mesh or knitted structures.

Additionally, any of the implantable members may also have a distal portion which is configured to be self-expanding or is a balloon-expandable stent-like structure to facilitate securement of the member to the vessel wall and inhibit migration. Alternatively, the securement mechanisms may comprise any number of configurations such as tissue penetrating barbs or hooks, etc. (bioabsorbable or otherwise). Additionally, such securement mechanisms may be placed along the length of the implantable device as so desired.

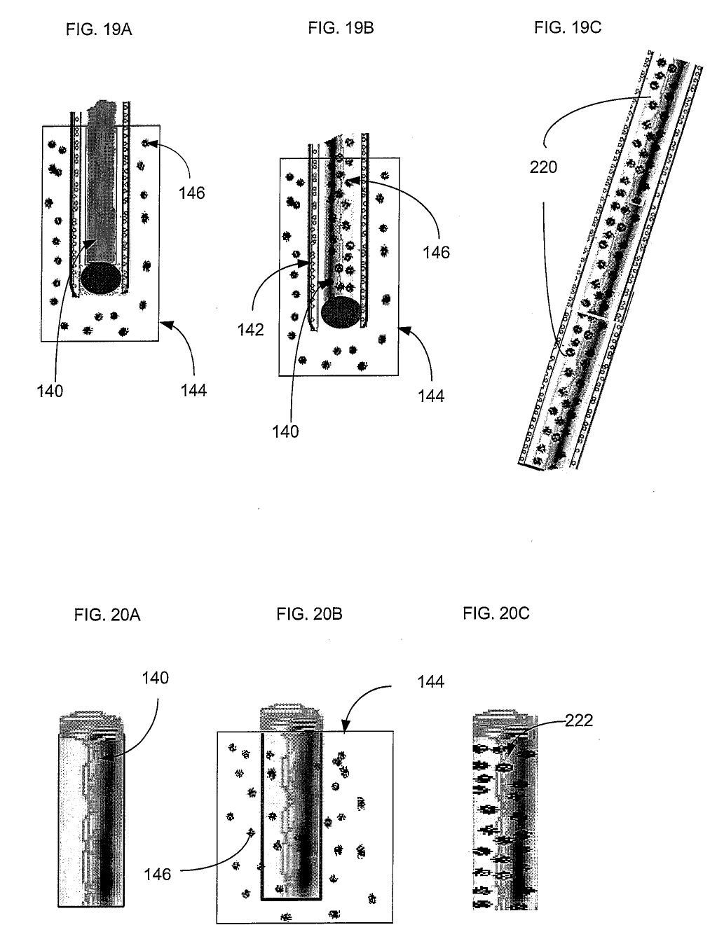

Additional examples for preparing the implantable device with a sclerosing agent are further described. For instance, FIGS. 19A and 19B illustrate one method of coupling of the biodegradable implant 140 with a sclerosing agent 146. As illustrated, FIG. 19A depicts a longitudinal cross-sectional view respectively of a biodegradable implant 140 placed in a perforated catheter sheath and immersed into a processing bath 144 filled with a sclerosing agent 146, as similarly described above.

FIG. 19B shows the biodegradable implant 140 immersed in processing bath 144 illustratively absorbing molecules of the sclerosing agent 146 through the pores or openings 142 defined over the sheath surface. The resulting implantable device 220 having the sclerosing agent 146 distributed throughout the implant is shown in FIG. 19C.

FIGS. 20A to 20C illustrate method for the process of coupling or applying the biodegradable implant 140 with a sclerosing agent 146 during its manufacturing process. As shown in FIG. 20B, biodegradable implant 140 may be immersed into processing bath 144 filled with sclerosing agent 146, prior to placement into the treatment catheter system. FIG. 20C illustrates biodegradable implant in a dried state 222 with sclerosing agent loaded therein, ready for placement into the treatment catheter system.

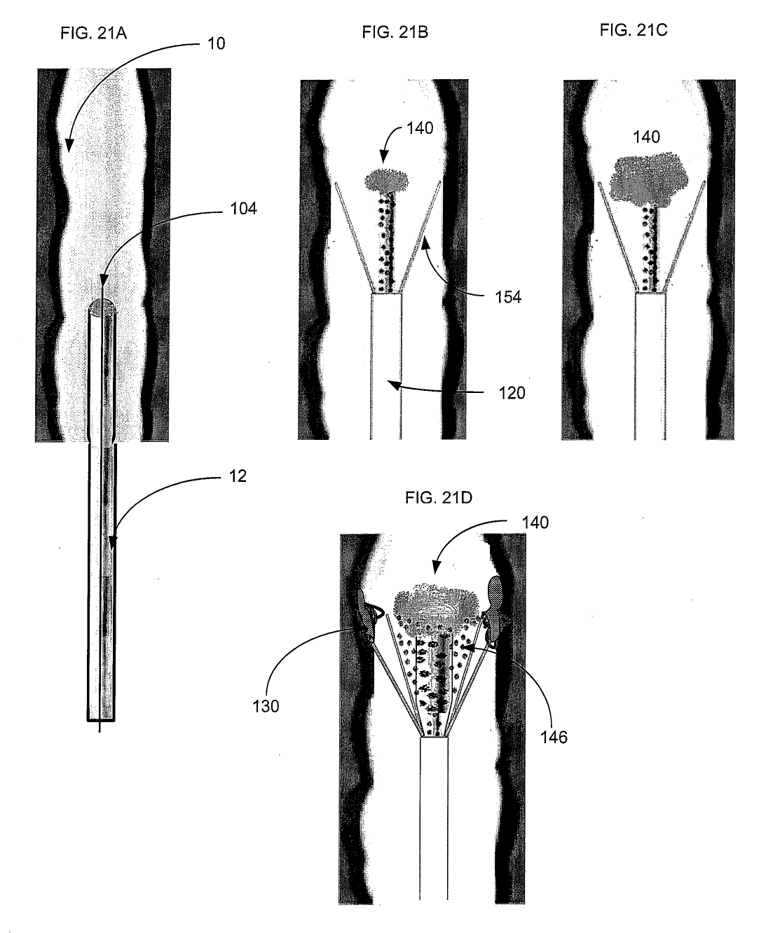

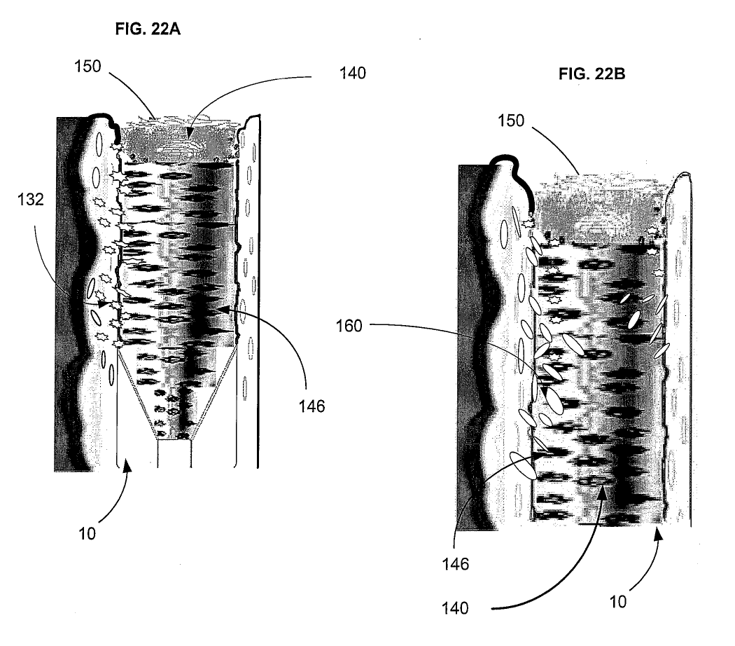

Once the implantable device has been prepared with the sclerosing agent, either before positioning within the catheter system or after placement within the catheter, it may be delivered and deployed within the vessel to be treated utilizing any of the methods described above. For instance, FIGS. 21A to 21D illustrate an additional method of introducing the treatment catheter system into the interior of the diseased vein 10 and deploying the implant 140 coupled with the application of simultaneous mechanical trauma to the vessel wall.

FIG. 21A depicts a longitudinal cross-sectional view respectively of a catheter system 12 introduced over the guidewire 104 into the deceased vein interior 10. Once desirably positioned, a distal end of the catheter sheath 120 may be unlocked, as described above, allowing for deflection of deflecting elements 154 and ejection of the implantable device 140, as shown in FIG. 21B.

As the implantable device 140 is exposed to the environment within the vessel, it may gradually expand inside of the vein, as shown in FIG. 21C. As the implantable device 140 expands, it may also begin to elute the sclerosing agent 146 into the surrounding tissue to further aggravate an inflammatory response 130, which may be initially activated by the mechanical trauma induced by deflecting elements 154. In this manner, the inflammation may be enhanced by the interaction of the inflamed vessel wall with sclerosing agent 146, as shown in FIG. 21D.

As the implantable device 140 further expands into contact against the tissue wall, the initiated cellular response may progress, as shown in FIG. 22A, the biodegradable implant 140 loaded with sclerosing agent 146 may become fully expended inside of the vein. Inflammation activated by the mechanical trauma coupled with the sclerosing agent may trigger an acute cellular response 132 characterized by the appearance of granulocytes, particularly neutrophils, in the tissues.

FIG. 22B depicts a longitudinal cross-sectional view respectively of biodegradable implant 140 now fully expended inside of the vein 10. Inflammation activated by the mechanical trauma coupled with sclerosing agent 146 progresses into a chronic cellular response 160. A characteristic of this phase of inflammation is the appearance of a mononuclear cell infiltrate composed of macrophages and lymphocytes. The macrophages are involved in microbial killing, in clearing up cellular and tissue debris, and they also seem to be very important in remodeling the tissues.

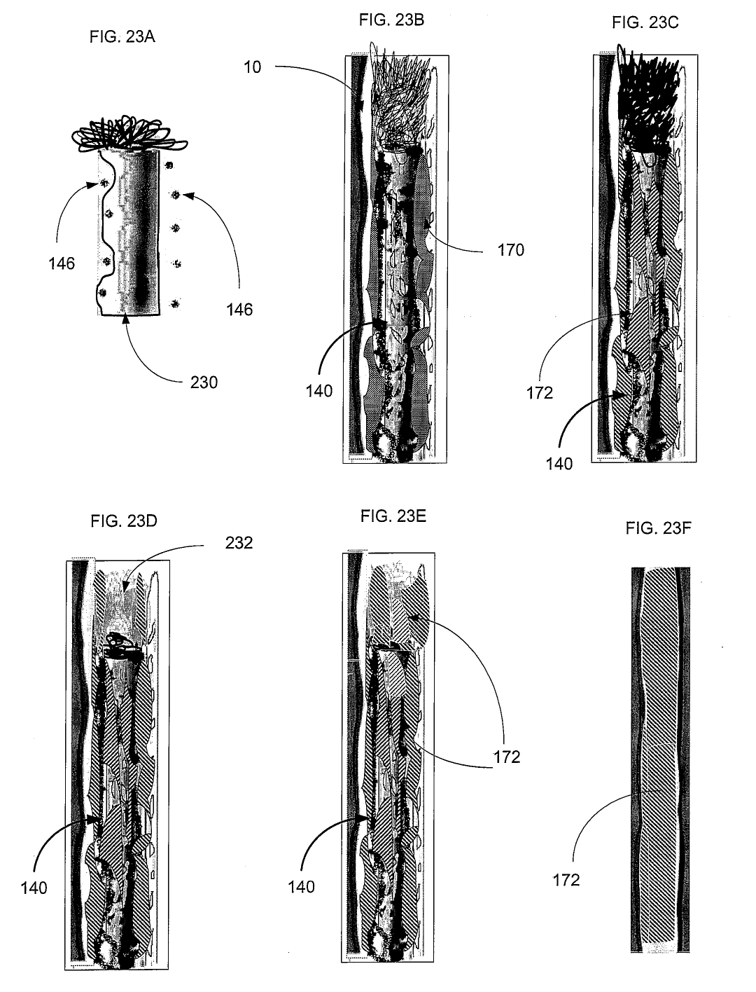

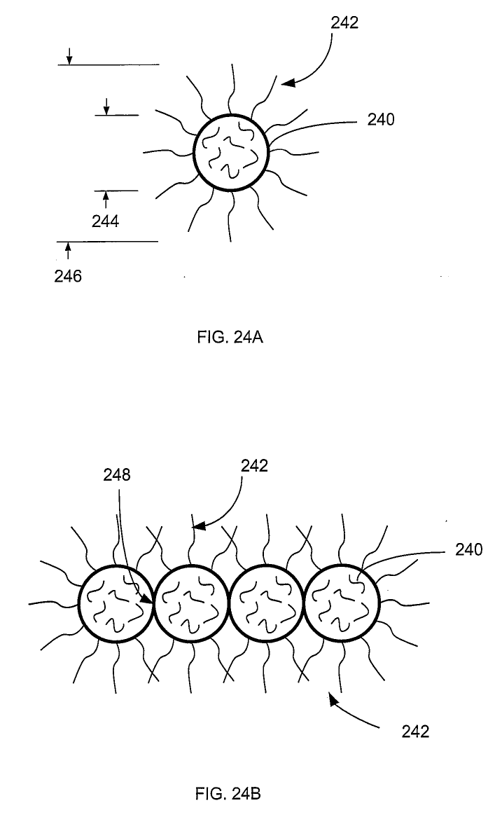

FIGS. 23A to 23F illustrate an example of the degradation process of the implanted device within the vessel and the resulting remodeled tissue wall in accordance with wound healing mechanisms. FIG. 23A depicts a longitudinal cross-sectional view respectively of a partially biodegraded implant 230 loaded with sclerosing agent 146. It also illustrates two different modes of a sclerosing agent release where the sclerosing agent 146 may be eluted from the implant via diffusion and via bioabsorption.

As the implant 140 continues to remain within the vessel 10, as shown in FIG. 23B, the inflamed vessel wall 10 begins the formation of granulation tissue 170. Granulation tissue has capillaries, fibroblasts, and a variable amount of inflammatory cells.

FIG. 23C illustrates a partially remodeled vessel wall during scar tissue formation 172. It also illustrates a relatively non-degraded exposed fibrous mesh component of the implant 140.

FIG. 23D illustrates a partially remodeled vessel wall during scar tissue formation 172. It also illustrates a gradual degradation of the exposed fibrous mesh component 232 of the implant 140.

FIG. 23E illustrates an almost completely remodeled vessel wall during scar tissue formation 172. It also illustrates a complete degradation of all the components of the implant 140.

FIG. 23F illustrates the resulting completely remodeled vessel wall filled with scar tissue 172. It also illustrates a significant shrinkage of the diseased vein.

Additional variations of the implantable device, including other shapes and configurations, may also be utilized aside from the tubular meshed structures shown and described above. For instance, FIG. 24A shows one variation that includes a biodegradable and/or bioresorbable occluding member 240. This occluding member 240 may be made of such materials to promote an inflammatory response, as described in detail above, and its architecture may be designed to accelerate conversion of thrombus to fibro-cellular tissue. To achieve high levels of inflammatory response, occlusion device 240 may be constructed of multiple layers of bioactive and bioresorbable materials and their copolymers, as above.

One example may include a multi-layer fibrous architecture having a first 244 and a second 246 outer surface. One or more fibers 242 may originate from the first surface 244 and are terminated in a spherical or cylindrical geometry to form the second outer surface 246.

One or more of the occlusion devices 240 may be attached to one another in series along attachment points 248 to form a chain of occlusion devices 240 having a length as desired, as shown in FIG. 24B.

Other variations of the occlusion devices may utilize a combination of a helical structure 250 for deployment within the vessel where the loops of the structure 250 may have one or more loop members 252, as shown in FIG. 25A, or strands 254, as shown in FIG. 25B, protruding through the open pitch of the helix 250.

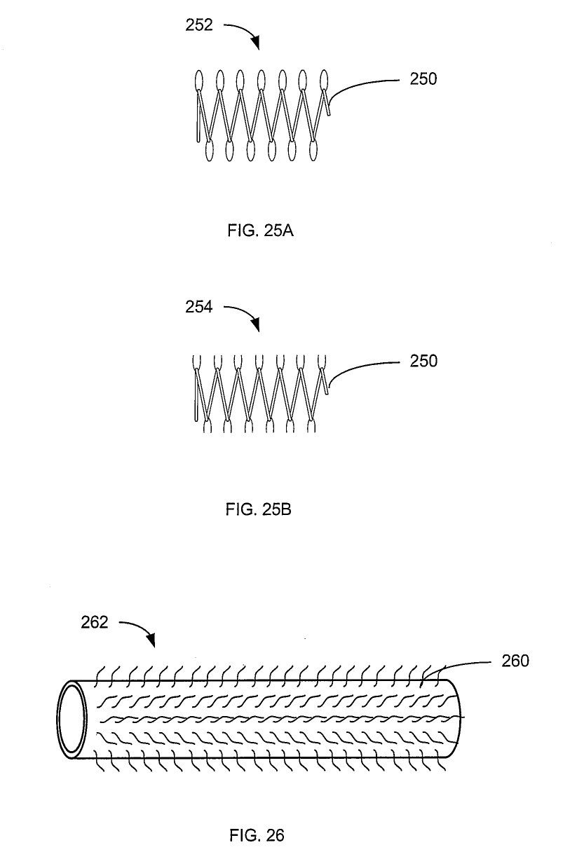

Yet another variation of an occlusion device may comprise an elongated hollow tubular segment 260, as described above, having one or more layers of fibers 262 attached to the inner and/or outer surfaces, as shown in FIG. 26.

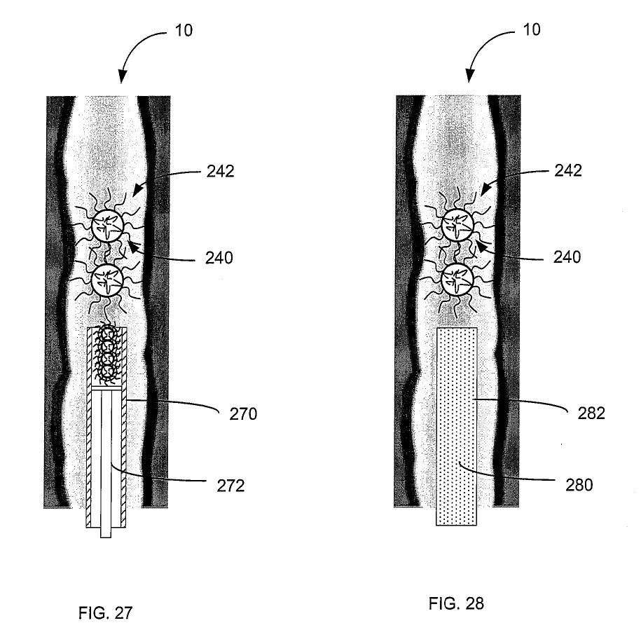

In use, a delivery catheter device 270 having one or more of the occlusion devices 240 pre-loaded therein may be advanced intravascularly adjacent to or proximate of the diseased tissue region within the vessel. A pusher mechanism 272 may be actuated to push or eject one or more of the occlusion devices 240 from the catheter 270 to expand within the vessel lumen into contact against the tissue wall, as shown in FIG. 27. The delivery catheter 270 utilized may include any of the delivery catheters described above and the occlusion devices 240 may also include any number of sclerosing agents applied thereto for enhanced tissue inflammation. The delivery catheter 270 may be pulled out proximally to cause controlled damage to the endothelium, while leaving the occluding member 240 implanted into the vein.

In another variation, the catheter 280 may define an abrasive outer surface 282 to further induce endothelial damage to the tissue wall. The entire catheter system may be rotated and the abrasiveness of catheter outer surface 282 may cause damage or the catheter 280 may be pulled proximally and rotated after or during ejection of the occlusion members to further enhance the inflammatory response of the vessel walls, as shown in FIG. 28. When the system is rotated contacting endothelial layer of the vein, this may enhance the controlled damage.

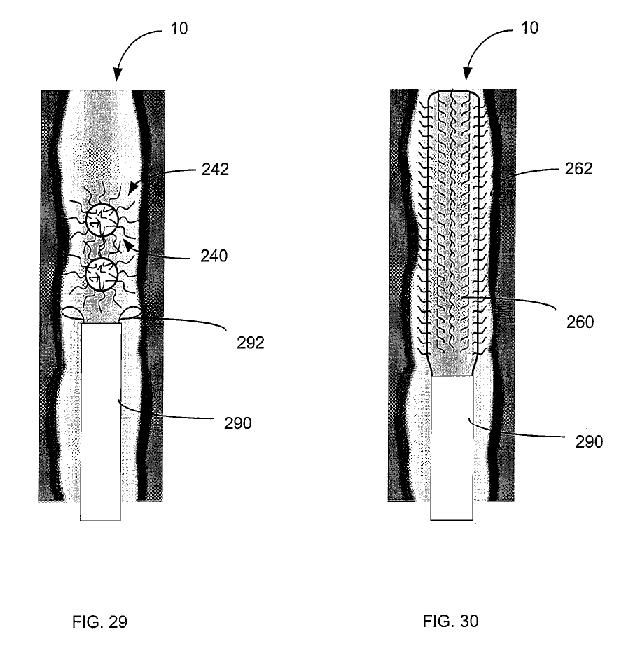

Yet another deployment method may include the use of energy application or mechanical trauma induction, e.g., via expandable members 292 extendable from catheter 290, as shown in FIG. 29. In such a variation, any of the above-mentioned energy modalities or mechanical mechanisms may be utilized with ejection and implantation of the occlusion members 240 and any of the sclerosing agent applications may also be applied to the occlusion members 240.

FIG. 30 shows another method of deploying the elongated hollow tubular segment 260 described above into the vessel. Prior to or during deployment of the segment 260, any of the inflammation inducing mechanisms described herein may be employed and the segment 260 may be infused with the sclerosing agent if so desired, as also described above.

The applications of the devices and methods discussed above are not limited to the treatment of insufficient veins but may include any number of further treatment applications. Other treatment sites may include areas or regions of the body such as arteries, airways, or other vessel walls within the body. Modification of the above-described assemblies and methods for carrying out the invention, and variations of aspects of the invention that are obvious to those of skill in the art are intended to be within the scope of the claims.

What is claimed is:

1. An apparatus for creating endovascular injury to tissue of a superficial, peripheral venous system, comprising:
an expandable outer member defining a lumen therethrough;
a porous layer disposed at least partially around a surface of the outer member; and
a sclerosing agent coupled within the surface of the outer member.

2. The apparatus of claim 1 further comprising an echogenic or radio-opaque marker attached to a distal segment of the surface or outer member.

3. The apparatus of claim 1 wherein the porous layer comprises foam layer or fibrous mesh.

4. The apparatus of claim 1 further comprising an elongate core member positioned within the lumen of the outer member.

5. The apparatus of claim 1 wherein the sclerosing agent is selected from the group consisting of alcohol, ethanol, chemotherapeutic agents, cytostatic agents, cytotoxic agents, sodium tetradecyl sulfate, Doxycycline, OK-432, saline and aethoxysclerol solutions, and combinations thereof.

6. The apparatus of claim 1 wherein outer member is adapted to apply the sclerosing agent infused within the porous layer against the tissue.

7. The apparatus of claim 1 wherein the outer member comprises an elongated balloon in fluid communication with an inflation/deflation lumen.

8. The apparatus of claim 7 wherein the balloon comprises an inner and outer balloon having different elasticity and compliance rates.

9. The apparatus of claim 7 wherein the balloon is comprised of one or more balloons arranged axially and in communication with a common inflation/deflation lumen.

10. The apparatus of claim 7 wherein the balloon is comprised of one or more balloons arranged axially and in communication with a separate corresponding inflation/deflation lumen.

11. The apparatus of claim 1 further comprising a guidewire insertable through the lumen.

12. The apparatus of claim 1 wherein the outer member is covered with multiple porous layers.

13. The apparatus of claim 1 wherein the outer member comprises an expandable metallic or polymeric structure.

14. The apparatus of claim 13 wherein the structure comprises a collapsed first diameter and an expanded second diameter.

15. The apparatus of claim 13 wherein outer member is comprised of a shape memory material.

16. The apparatus of claim 1 wherein the outer member is constrained via a pulling mechanism.

17. The apparatus of claim 16 further comprising a pullwire or draw string coupled to the outer member.

18. The apparatus of claim 1 wherein the porous layer is selected from the group consisting of silicone, ePTFE, acrylic copolymer, polyurethane, polyethylene, polyamide, polyamide, PEEK, PET, HDPE, PVDF, Pebax, PVDF, Teflon, polyurethane and/or their copolymers.

19. The apparatus of claim 1 wherein the porous layer is formed from a rolled flat sheet secured onto the outer member.

20. The apparatus of claim 1 wherein the porous layer is formed from a hollow tube secured onto the outer member.

21. The apparatus of claim 1 wherein the porous layer is formed from a coating applied onto the outer member.

22. The apparatus of claim 1 further comprising an elongate sheath configured for placement over the outer member.

23. The apparatus of claim 22 wherein the sheath defines a plurality of porous or openings thereupon.

24. A system for generating an environment internal to the venous system that causes obliteration of a diseased vein over a time period, comprising:
an expandable outer member defining a lumen therethrough;
a porous layer disposed at least partially around a surface of the outer member;
a sclerosing agent coupled with the outer member; and
a biodegradable scaffold removably positioned within the lumen.

25. The system of claim 24 wherein the porous layer is selected from the group consisting of Silicone, Expanded Polytetrafluoroethylene, acrylic copolymer, polyurethane, polyethylene, polyamide, polyimide, Polyetheretherketone, Polyethylene terephthalate, High Density Polyethylene, Polyvinylidene Fluoride, Pebax, Polytetrafluoroethylene, polyurethane and their copolymers.

26. The system of claim 24 wherein the porous layer is formed from a rolled flat sheet secured onto the outer member.

27. The system of claim 24 wherein the porous layer is formed from a hollow tube secured onto the outer member.

28. The system of claim 24 wherein the porous layer is formed from a coating applied onto the outer member.

29. The system of claim 24 wherein the biodegradable scaffold is comprised of a polymeric material selected from the group consisting of polylactic acid, polyglycolic acid and their copolymers, polydioxanone, polycaprolactive, vitronectin, polycarbonates, polyanhydrides, fibronectin, lamin, fibrinogen, polyhydroxybutyrate, hydroxyvalerate copolymers, hyaluronic acid, cellulose, polyhyaluronic acids, casein, collagen, gelatin, gluten, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), endothelial growth factors, ion implants, and combinations thereof.

30. The system of claim 24 further comprising a core member positioned through the detachable biodegradable scaffold.

31. The system of claim 30 wherein the core is comprised of a helical structure having loops or fibers attached thereto.

32. The system of claim 30 wherein the core member is non-porous.

33. The system of claim 24 wherein the biodegradable scaffold is coupled with the sclerosing agent

34. The system of claim 24 wherein the sclerosing agent is selected from the group consisting of cytostatic agents, cytotoxic agents, alcohol, chemotherapeutic agents, ethanol, Doxycycline, sodium tetradecyl sulfate, saline and aethoxysclerol solutions, and combinations thereof.

35. The system of claim 24 further comprising a sheath for placement over the outer member.

36. The system of claim 35 wherein the sheath defines a plurality of pores or openings thereupon.

37. The system of claim 24 further comprising a delivery catheter connected to a proximal end of the outer member.

38. The system of claim 37 wherein a proximal segment of the delivery catheter is comprised of a material selected from the group consisting of Polyetheretherketone, Polyethylene terephthalate, High Density Polyethylene, Polyethylene, Polyimide, Polyamide, Pebax, Polyvinylidene Fluoride, Polytetrafluoroethylene, Polyurethane and copolymers thereof, and combinations thereof.

39. The system of claim 24 further comprising an echogenic or radio-opaque marker attached to a distal segment of the outer member.

40. The system of claim 24 wherein a distal segment of the detachable biodegradable scaffold comprises a fibrous mesh.

41. The system of claim 24 wherein a distal segment of the detachable biodegradable scaffold has a slower absorption rate than a proximal portion of the biodegradable scaffold.

42. The system of claim 24 wherein a distal segment of the detachable biodegradable scaffold is comprised of a non-absorbable polymeric or metallic material.

43. The system of claim 42 wherein the non-absorbable polymeric or metallic material is selected from the group consisting of polyester fibers, Expanded Polytetrafluoroethylene, Polytetrafluoroethylene, Platinum, Gold, stainless steel, Nickel-Titanium alloys, and combinations thereof.

44. The system of claim 24 wherein a distal segment of the detachable biodegradable scaffold is configured to be secured to a vessel wall.

45. The system of claim 44 wherein the distal segment comprises a self-expanding or balloon-expandable structure or penetrating hooks or barbs.

46. The system of claim 44 further comprising additional securement member positioned along a length of the biodegradable scaffold.

47. The system of claim 24 wherein the biodegradable scaffold comprises:
a multi-layer fiber construction which defines an internal surface and an external surface; and
a plurality of fibers which originate from the internal surface such that free ends of each fiber forms the external surface of the biodegradable scaffold.

48. The system of claim 24 wherein the detachable biodegradable scaffold has a geometry configured to promote and accelerate a scarring response from a vessel wall.

49. The system of claim 24 wherein the detachable biodegradable scaffold comprises a helical structure having a plurality of fibers protruding therefrom.

50. The system of claim 24 wherein the detachable biodegradable scaffold comprises a hollow tubing having a plurality of fibers protruding from its outer and inner surfaces.

51. A system for generating an environment internal to a venous system that causes obliteration of a diseased vein over a time period, comprising:
an expandable outer member defining a lumen therethrough;
a biodegradable member removably disposed at least partially around a surface of the outer member; and
a sclerosing agent coupled with the biodegradable detachable member

52. The system of claim 51 wherein the biodegradable detachable member has a multi layer porous membrane architecture which defines an internal surface and an external surface.

53. The system of claim 51 wherein the biodegradable detachable member has a multi layer comprising of porous and nonporous membrane architecture.

54. A system for treatment of venous insufficiency comprising:
an outer member defining a lumen therethrough;
a biodegradable scaffold removably positioned within the lumen; and
at least one deflectable member affixed to the outer member

55. The system of claim 54 wherein the deflectable members generate mechanical trauma to the interior of the vessel wall.

56. The system of claim 54 wherein the deflectable members deliver thermal energy and generate trauma to the interior of the vessel wall.

57. The system of claim 54 wherein the detachable biodegradable scaffold is coupled with a sclerosing agent.
